(19) United States
(12) Patent Application Publication
(10) Pub. No.: US 2003/0059816 A1
(43) Pub. Date: Mar. 27, 2003

(54) Methods for identifying racemases

(75) Inventors: Ian Fotheringham, Schaumburg, IL (US); Sylvain Royer, Edinburgh (GB)

Correspondence Address: Patrick G. Gattari, McDonnell Boehnen Hulbert & Berghoff, 32nd Floor, 300 S. Wacker Drive, Chicago, IL, 60606, US

(73) Assignee: Thermogen, Inc.

(21) Appl. No.: 10/211,695

(22) Filed: Aug. 2, 2002

Related U.S. Application Data
(60) Provisional application No. 60/310,209, filed on Aug. 3, 2001.

(51) Int. Cl. C12Q 1/68; C12Q 1/34; C12Q 1/04

(52) U.S. Cl. 435/6; 435/18; 435/34

(57) ABSTRACT

This invention describes methods for the identification of enzymes from the enolase superfamily with racemase activity for industrial applications. The methods comprise obtaining a micro-organism which requires the enzyme with the acquired racemase activity to grow in a selective medium. The micro-organism is transformed with a gene encoding the enzyme, grown in the selective medium supplemented with a substrate of the racemase, and one or more micro-organism(s) that is able to grow in the supplemented selective medium is then isolated, thereby identifying the enzyme with the acquired activity. The methods further comprise genetic alterations of the genes that encode enzymes resulting in the acquired racemase.

### FIELD OF THE INVENTION

The invention is in the field of methods to identify enzymes with novel activities. In particular, the methods are directed to the identification of enzymes with N-acyl-amino acid and acylamine racemase activities derived from microbial gene libraries. The invention also relates to methods of creating new racemases by directed evolution from related enzyme activities.

### BACKGROUND OF THE INVENTION

An earlier study identified a racemase in an Amycolatopsis species which displayed relaxed substrate specificity upon N-acylated amino acids but no activity towards the free amino acid equivalents (Appl. Microbiol. Biotechnol. 42:884-889). The enzyme showed activity upon a broad range of N-acylated amino acid substrates, particularly methionine and phenylalanine. Further work was carried out upon this enzyme to explore its potential in application for the production of enantiomerically pure amino acids. A second enzyme with racemase activity was described in Streptomyces atratus Y-53 (Tokuyama, S. Journal of Molecular Catalysis B: Enzymatic 12 (2001) 3-14). The racemase activity was shown to be a secondary activity of this enzyme which is primarily a dehydratase and perhaps more significantly a member of the enolase superfamily of enzymes which abstract an (x-proton from a carboxylate substrate. If this enzyme is used in conjunction with an enantioselective amidase or acylase, it offers the possibility to establish a dynamic kinetic resolution process for producing a single amino acid isomer from a racemic mixture of particular acylated amino acid substrates.

The enzyme was later characterized as a dehydratase, O-succinyl benzoate synthase, and not as a racemase. The lower level of racemase activity was a side reaction that occurs in the presence of N-acylamino acid substrates. Presently, there is no evidence to support any selective pressure or advantage for the organism to have evolved this activity.

Although examples of its potential use in an applied bioprocess have been described at low substrate concentration (<50 mM), the enzyme has not been developed for commercial use. This is partly due to the highly competitive market for low cost amino acid production that favors methods such as fermentative production and alternative resolution methods employing enzymes such as hydantoinase. Additionally, these racemases have other characteristics that significantly limit their use as biocatalyst. These characteristics are low turnover and substrate mediated inhibition at concentrations exceeding 50 mM.

An enzyme with optimized racemase activity that possesses low substrate inhibition, stability, and reusability would have important industrial relevance. Moreover, if the activity could be expanded by directed evolution to accept acylated amines instead of N-acylamino acids, its industrial significance could be very useful in the area of chiral amines. Chiral amines are key intermediates in the pharmaceutical industry and are also produced by the chemical industry for synthesizing industrial polymers, for example, 1-phenylethylamine.

Hydrolase-based resolution processes are a principle enzymatic route currently employed in the synthesis of chiral amines and no broadly applicable enzymatic methods exist for the direct enantioselective synthesis of chiral amines. More than 60% of enzyme reactions conducted by for commercialization are enantioselective or regioselective resolution processes which utilize hydrolase enzymes such as acylases, esterases or lipases. In most cases the yield is limited to a maximum of 50% due to the unreacted isomer. To exceed this yield, complex and expensive recycling processes are required, generally involving many chemical steps. In many cases, and for economic reasons, the processes do not recycle the unreacted isomer which is discarded, creating a further problem in waste disposal.

The ability to increase the yield of such processes towards 100% through in situ racemisation of substrate is a major goal in applied biocatalysis. Methods to identify novel enzymes to use in methods for dynamic kinetic resolution are urgently needed. The potential of developing a substrate-specific racemase enzyme to use in conjunction with the well established hydrolases such as acylases, esterases or lipases is within the reach of available technologies especially since enzymes with low racemase activity have been shown to exist.

Enzyme families comprise enzymes which possess peptide sequence conservation and/or conservation of enzyme structure and catalyze a similar reaction. Superfamilies comprise multiple families of enzymes which catalyze different reactions but show sequence and/or structural conservation and common reaction features. The enolase superfamily comprises enzymes which are related by sequence and structure and their common ability to catalyze the abstraction of the α-proton of a carboxylic acid to form an enolic intermediate. This family is designated by the name of the family member most central to living organisms, enolase (Biochemistry (1996) 35, 16489-16501″ Babbitt, P. C. et al.).

Although earlier studies showed that racemases are not often ideal for biocatalysis due to slow turnover and substrate inhibition, other features common to racemases make them appropriate as biocatalysts. Primarily, there is frequently no requirement for cofactor recycling or reducing equivalents and they can be used in cell-free form or in an immobilized form. Secondly, racemases are cytosolic enzymes produced from a single gene which simplify their study and isolation of additional genes encoding racemases.

### SUMMARY OF THE INVENTION

This invention describes methods for the identification of enzymes from the enolase superfamily with racemase activity. The methods comprise obtaining a micro-organism which requires the enzyme with the acquired racemase activity to grow in a selective medium. The micro-organism is transformed with a gene encoding the enzyme, grown in the selective medium supplemented with a substrate of the racemase, and one or more micro-organism(s) that is able to grow in the supplemented selective medium is then isolated, thereby identifying the enzyme with the acquired activity. The methods further comprise genetic alterations of the genes that encode enzymes resulting in the acquired racemase. Other acquired activities as a result of these genetic alterations can include but are not limited to enolase, cycloisomerase, and O-succinyl benzoate synthase. Two particular acquired racemase activities are N-acylamino acid racemase or acylamine racemase. Other racemase activities can be identified by using an appropriate substrate and establishing the selection pressure to evolve specific racemase activity. For example, by using a single enantiomer of an N-acylamino acid as substrate, an enzyme that acquires N-acyl-amino acid racemase activity can be identified using the methods of this invention. Similarly, using a single enantiomer of an acylated amine as substrate, an enzyme with acylamine racemase activity can be identified.

The gene encoding an enzyme with N-acylamino acid racemase activity can be derived from a gene library prepared from the chromosome of a micro-organism or from the group of genes that encode members of the enolase enzyme superfamily. Micro-organisms which can be used include but not limited to Bacillus, Actinomyces, Rhodococcus, Escherichia, Salmonella, Klebsiella, and Pseudomonas. The gene library is subjected to random genetic mutation by passage through a mutator strain of E. coli. such as XL1-Red. Another way to produce random mutations is using error prone PCR. The gene library can also be subjected to site-directed mutagenesis based on the conservation of amino acid sequence, structure or enzymatic mechanism. The micro-organism itself contains one or more mutations which create one or more amino acid auxotrophies and a deficiency in D-amino acid oxidase activity which prevents alternative means of growth.

This invention also describes a recombinant bacterial strain that is auxotrophic for a specific enantiomer of one or more amino compounds. This bacterial strain is able to preferentially hydrolyze the acyl derivatives of amino compounds for which the bacterial strain is auxotrophic and to couple this hydrolysis reaction with an enzyme having racemase activity. The enzyme having racemase activity is expressed from a gene carried on a vector. The racemase encoded by the gene is N-acylamino acid racemase but any racemase encoded by a gene can be inserted into the vector. The result of this coupling is that the bacterial strain is able to produce a single enantiomer of the amino compounds from an acyl racemic mixture of the amino compounds, thus enabling the bacterial strain to grow in selective medium that lacks the specific enantiomer of the amino compounds for which it is auxotrophic.

A method to enantioselectively produce an L-amino acid or a D-amino acid comprising combining a hydrolytic enzyme with the racemase identified according to claim 1. Another aspect of the invention is the use of an enzymatic process for enantioselectively producing an L-amino acid or a D-amino acid comprising combining a hydrolytic enzyme with a racemase identified according to the method of this invention. In one variation of the enzymatic process, one or more enzymes are immobilized on a support.

In one specific aspect of the invention, a screening methods is designed to identify novel racemases derived by mutation of one or more genes encoding a member or members of the enolase superfamily of enzymes. These screening methods comprise mutation of a gene and transformation of a bacterial strain with a vector bearing the mutated gene. The bacterial strain requires the product of novel racemase activity for growth in selective media that lacks this product but is supplemented with the substrate of the racemase. By selecting the bacterial strains that are capable of growing in the supplemented selective media that lacks the product of the racemase, novel racemases can be identified.

### BRIEF DESCRIPTION OF THE FIGURES

FIG. 1 illustrates the procedure to identify a bacterial cell such as an appropriate strain of E. coli which can hydrolyze particular N-acyl-L-amino acids such as N-acetyl or N-phenylacetyl amino acids to provide free amino acid to relieve a specific amino acid auxotrophy enabling it to grow in selective media. The establishment of the amino acid requirement is achieved using L-amino acid auxotrophs, for example, a pheA mutant requiring exogenous L-phenylalanine or a metB mutant requiring exogenous L-methionine. This involved testing for hydrolysis of N-acyl-L-amino acid as the only supply of amino acid as well as determining if it is necessary to supply exogenous hydrolase. A variety of N-acyl groups can be used.

FIG. 2 illustrates the procedure by which a bacterial strain auxotrophic for an amino acid and able to hydrolyze N-acyl derivatives of that amino acid can be used to identify enzymes with N-acylamino acid racemase activity. The establishment of the amino acid requirement was done using L-amino acid auxotrophs, for example, pheA mutant requiring L-phenylalanine, and using conditions for N-acyl-L-amino acid hydrolysis. The bacterial strain is unable to utilize N-acyl D-amino acids due to a mutation in the dadA gene encoding D-amino acid oxidase. Bacterial strains that are able to grow contain a racemase that is able to racemisize N-acyl-D-amino acid to relieve the L-amino acid auxotrophy. A variety of N-acyl groups can be used.

FIG. 3 illustrates the procedure by which an auxotrophic bacterial strain requiring an (S) amine and able to hydrolyze acylated (S)-amines but not acylated (R)-amines can be used to identify enzymes with acylamine racemase activity. This was done by establishing a chiral amine requirement and using conditions for acyl-(S)-amine hydrolysis. The auxotrophic bacterial strain is able to utilize acyl-(S)-amine to satisfy the amine requirement but not the acyl-(R)-amine. Bacterial strains are selected that are able to grow in media supplemented with only acyl-(R)-amine. A variety of acyl groups can be used.

FIG. 4 illustrates the procedure by which a strain requiring an (R)-amine and able to utilize acyl-(R)-amines to satisfy the amine requirement but not acyl-(S)-amines can be used to identify enzymes with acylamine racemase activity. The was done by establishing a chiral amine requirement and using conditions for acyl-(R)-amine hydrolysis. The auxotrophic bacterial strain can utilize acyl-(R)-amine but not the acyl-(S)-amine. Bacterial strains are selected that are able to growth in media supplement with only acyl-(S)-amine. A variety of acyl groups can be used.

FIG. 5 illustrates the procedure by which a strain requiring an L-amino acid is used to identify enzymes with acylamine racemase activity. The strain is able to hydrolyze acyl-(R)-amines but not acyl-(S)-amines and can be used in conjunction with an omega amino acid aminotransferase specific for the (R)-isomer of an amine to transfer an amino group to a required amino acid. This is done by establishing an L-alanine requirement and using conditions for acyl-(R)-amine hydrolysis. FIG. 5. Also shows how a strain can be used with an enantioselective oxidase or aminotransferase to liberate ammonia as a source of nitrogen for strain growth The auxotrophic bacterial strain can utilize acyl-(R)-amine but not the acyl-(S)-amine. Bacterial strains are selected that are able to growth in media supplemented with only acyl-(S)-amine. A variety of acyl groups can be used

### DETAILED DESCRIPTION OF THE INVENTION

This invention relates to methods for the identification of novel N-acylamino acid and acylamine racemases from microbial gene libraries. Furthermore, the invention also relates to ways in which using procedures of directed evolution starting with related enzyme, enzymes with new racemases activities could be identified. Related enzymes are members of the enolase superfamily which have been identified and their gene sequences published. It is likely that many members can be identified through sequence conservation alignment using the vast data available for microbial genes and in genome banks. The invention makes uses of selective methods based on microbial mutants deficient in specific amino acids (auxotrophs) in order to identify additional enzymes with N-acylamino acid racemase activity. In one embodiment of the invention, specific substrates are used to select for specific properties in the engineered enzymes. These methods are used to select acylamine racemase and N-acylamino acid racemase activities from libraries of variants derived from known enzymes and their homologues. Another embodiment of the invention involves the use of recombinant strains bearing amine oxidases or omega aminotransferase activities as nitrogen scavengers to rescue mutants as a method to select novel racemase activities.

The first step of the invention involves constructing or obtaining from appropriate sources (for example, ATCC or CGSC) bacterial strains which are deficient in their ability to synthesize (that is, auxotrophic for) one or more amino acids of specific chiral configuration and which when grown on a minimal medium such as M9 require that the growth medium to be supplemented with amino acid(s) for which they are auxotrophic.

Once an auxotrophic strain is obtained, it is tested for its ability to hydrolyze N-acyl derivatives of the required amino acids by testing for growth on minimal media such as M9 which is supplemented with an N-acylated derivative of the amino acid instead of the free amino acid. To enhance the hydrolytic activity, auxotrophic strains can be transformed with a plasmid vector which expresses a gene encoding an amidase or acylase, for example, the ama gene of B. stearothermophilus NCIB 8224 (Applied and Environmental Microbiology 1993 59:3878-3888).

The next step in the identification of novel racemases according to methods of this invention is to test the viability of the auxotrophic strain in minimal medium supplemented with the N-acylated derivative of the opposite isomer of the required amino acid(s). Cells that are able to survive possess an endogenous racemase activity that is able to convert the opposite isomer to the N-acylated derivative of the amino acid isomer that the cell requires for survival. Strains which are unable to utilize such N-acylated-amino acid derivatives of the opposite isomer but are able to hydrolyze and utilize the N-acylated-amino acid derivatives of the required isomer form the basis of an in vivo selection to identify novel N-acylamino acid racemases

In one particular embodiment of the invention, the novel racemase is encoded in a gene fragment which is introduced into the auxotrophic strain by plasmids or phage vectors. The gene fragments maybe be derived from a random population of gene fragments.

An in vivo selection approach is a method to identify a particular enzyme activity or enzyme variant by the fact that it confers a property onto a living organism in vivo which allows identification (selection) of the organism carrying the desired enzyme/variant from all others around it when grown under particular conditions that allows for that selection. Such properties could be resistance to a toxic compound or restoration of a growth deficiency. It is distinguished from an in vitro approach which requires extraction of the biological material from the cell and external assay for detection. Its advantage lies in the simplicity of application and high numbers of organisms which can be readily screened.

The gene fragments can be derived from gene libraries prepared from heterologous organisms or genes known to encode related activities which can be introduced into the auxotrophic organism. The gene fragments can be used in their wild-type form or be subjected to mutagenesis using a variety of methods including but not limited to rational site-directed mutagenesis and random mutagenesis. Random mutagenesis can be performed using chemical mutagens, irradiation or biological methods. Biological methods of random mutagenesis includes but not limited to mutator strains such as XL1-Red (Stratagene, La Jolla) and error prone PCR using a kit such as Diversify (ClonTech). Sources of organisms, random gene fragments and sources of genes encoding related enzyme activities can be obtained from the scientific literature, culture collection databases or and from online resources such as DNA databases, for example, GenBank.

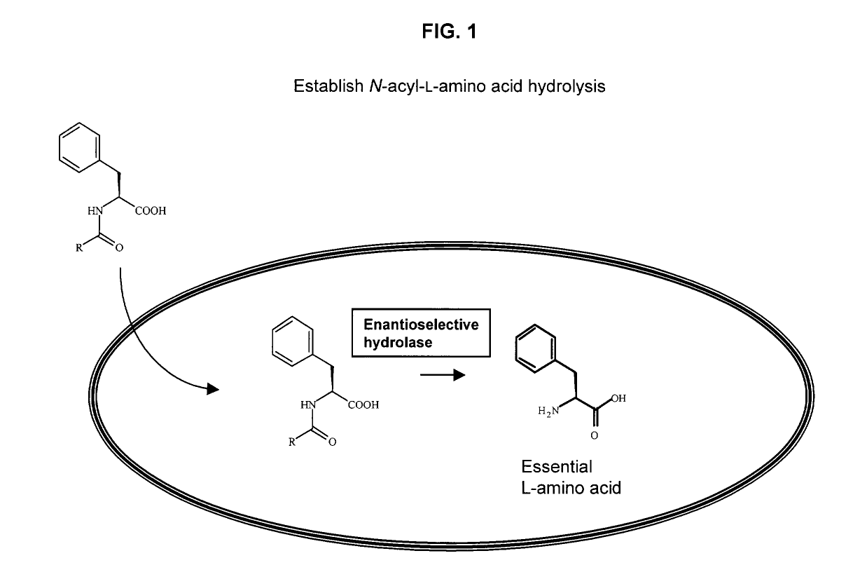

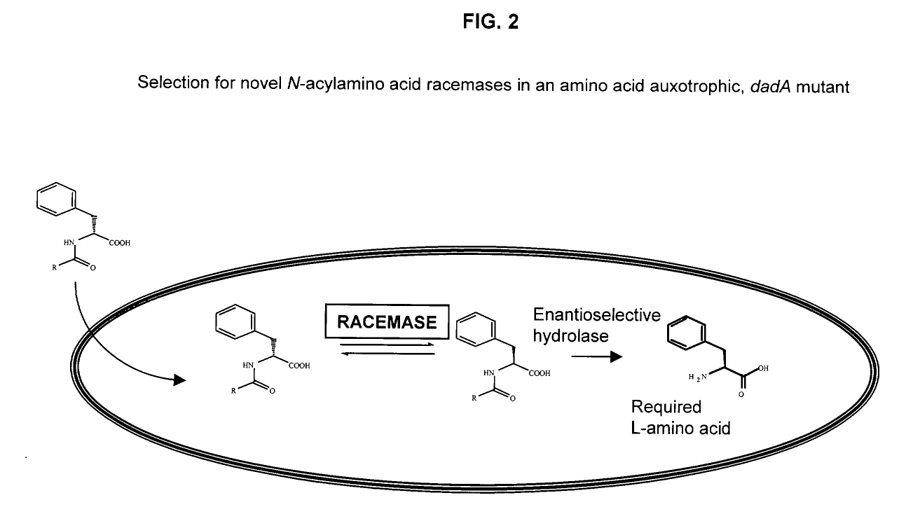

The validity of microbial-based selection methods for the identification of novel N-acylamino acid racemases can be tested by adding to the growth medium N-acyl derivatives of the enantiomer of the amino acid for which the cell is auxotrophic. When supplemented with the N-acylamino acid the strain is able to hydrolyze the N-acylamino acid to release the free amino acid and relieve the auxotrophy. When supplemented with the N-acylamino acid of the opposite isomer, the strain requires a racemase to first convert the N-acylamino acid to the correct isomer of the N-acylamino acid to relieve the auxotrophy. For example, a strain auxotrophic for an L-amino acid and grown in medium supplemented with the N-acyl-L-amino acid are able to hydrolyze the N-acyl-L-amino acid to release the free amino acid and relieve the auxotrophy. When the medium is instead supplemented with the N-acyl-D-amino acid, the strain requires a racemase to convert the N-acyl-D-amino acid to the N-acyl-L-amino acid to relieve the auxotrophy. (FIGS. 1 and 2). Even if the strain can hydrolyze the N-acyl-D-amino acids it is unable to use the free D-amino acid to relieve the auxotrophy due to the mutation in dadA encoding D-amino acid oxidase.

To test the validity of the methods described in this invention, a gene encoding a known N-acyl amino acid racemase can be isolated and cloned onto a vector capable of expressing the gene in a an auxotrophic strain such as that described above. For example the gene encoding N-acylamino acid racemase from Amycolatopsis species TS-1-60 can be isolated from the chromosomal DNA of the host strain using standard methods of chromosomal DNA preparation and by amplifying the N-acylamino acid racemase encoding gene by PCR using oligonucleotide primers derived from the gene sequence as described in Genbank under accession number 2147746. Activity of this enzyme can be demonstrated in a recombinant E. coli host strain transformed with a plasmid vector, which expresses this gene from a regulatory region functional in E. coli. Mutagenesis experiments using the methods described above can also be conducted to identify variants of the enzyme which display altered N-acylamino acid racemase activity such as altered substrate specificity or increased stability.

In another embodiment of the invention, variant enzymes can be selected that exhibit acylamine racemase activity. Such activity can be identified using a modification of the above methods described above. The first step would be to establish conditions whereby an organism is dependent upon a specific enantiomer of a chiral amine as a source of nitrogen for the growth of the organism, that is, auxotrophic for that specific chiral amine. This is achieved by growing the auxotrophic organism in a minimal medium such as a modified M9 in which all existing nitrogen sources are replaced the opposite isomer of the amine. Growth is tested to determine if cells can grow in the presence of the opposite isomer as sole nitrogen source. Growth on a chiral amine as a sole nitrogen source could be enhanced by introducing into the auxotrophic strain a heterologous gene encoding an amine oxidase or amine aminotransferase which is expressed either from a plasmid or phage vector. For example, the omega amino acid aminotransferase of P. aeruginosa can be used to enhance growth.

In another embodiment, the omega amino acid aminotransferase introduced into the auxotrophic strain can be used to synthesize an essential amino acid such as L-alanine or D-alanine such that growth of the organism was dependent upon the single enantiomer of a chiral amine as a precursor for L-alanine or D-alanine biosynthesis. The ability of the strain to use other means to synthesize D-alanine essential for cell wall biosynthesis can be eliminated by mutation of the genes alr and dadX which encode the alanine racemases of E. coli. Additionally the activity of the alanine racemases can be sufficiently inhibited to prevent cell growth by the addition to the growth medium of an inhibitor such as beta-chloro-pyruvate or beta-chloro-D-alanine.

After identifying the appropriate amines which can be used as nitrogen sources, the auxotrophic strains are then tested for their ability to use acyl derivatives of each enantiomer of the amine. This is done by testing for growth on a minimal medium such as a modified M9 which is supplemented with an acylated derivative of either enantiomer of the amine instead of the underivatized amine. Growth under such conditions requires that the organism be able to take up and hydrolyze the acyl derivatives of the particular required enantiomer of the amine in the medium. To enhance this hydrolytic activity, the auxotrophic strains can be transformed with a plasmid vector which expresses a gene encoding an amidase or acylase activity such as the ama gene of B. stearothermophilus.

Strains that are able to utilize an acyl derivative of one enantiomer of the chiral amine as sole nitrogen source for growth are then tested for their inability to use an acyl derivative of the opposite enantiomer of the required amine as a source of the required amine. Growth under these conditions would require an endogenous racemase specific for the required amine or for the acylated derivative.

Strains which are unable to utilize such derivatized amines of the opposite enantiomer as sole nitrogen source form the basis of an in vivo selection to identify acyl amine racemase activities from a random population of gene fragments which can be introduced to the strain cloned on plasmid or phage vectors.

The gene fragments can be derived from gene libraries prepared from heterologous organisms or genes known to encode related activities which can be introduced into the auxotrophic organism. The gene fragments can be used in their wild-type form or be subjected to mutagenesis using a variety of methods including but not limited to rational site-directed mutagenesis and random mutagenesis. Random mutagenesis can be performed using chemical mutagens, irradiation or biological methods. Biological methods of random mutagenesis includes but not limited to mutator strains such as XL1-Red (Stratagene, La Jolla) and error prone PCR using a kit such as Diversify (ClonTech). Sources of organisms, random gene fragments and sources of genes encoding related enzyme activities can be obtained from the scientific literature, culture collection databases or and from online resources such as DNA databases, for example, GenBank.

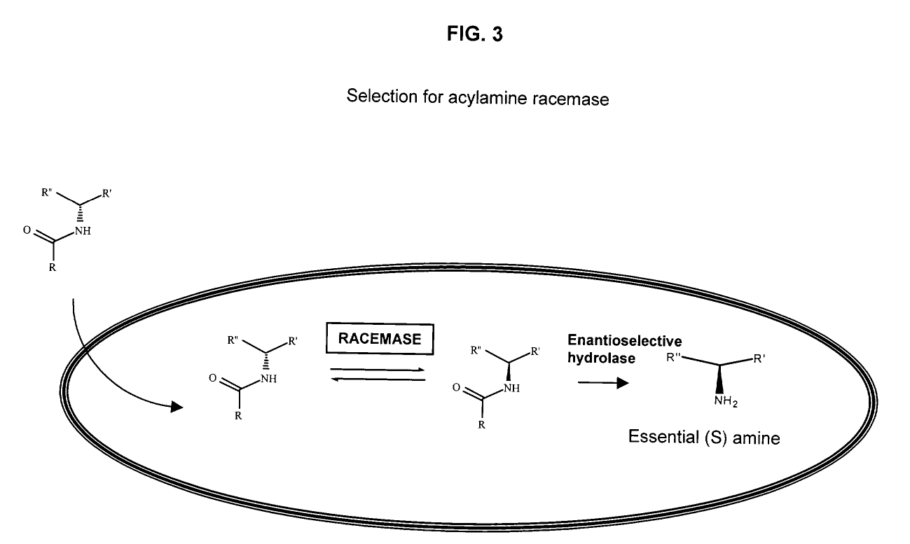

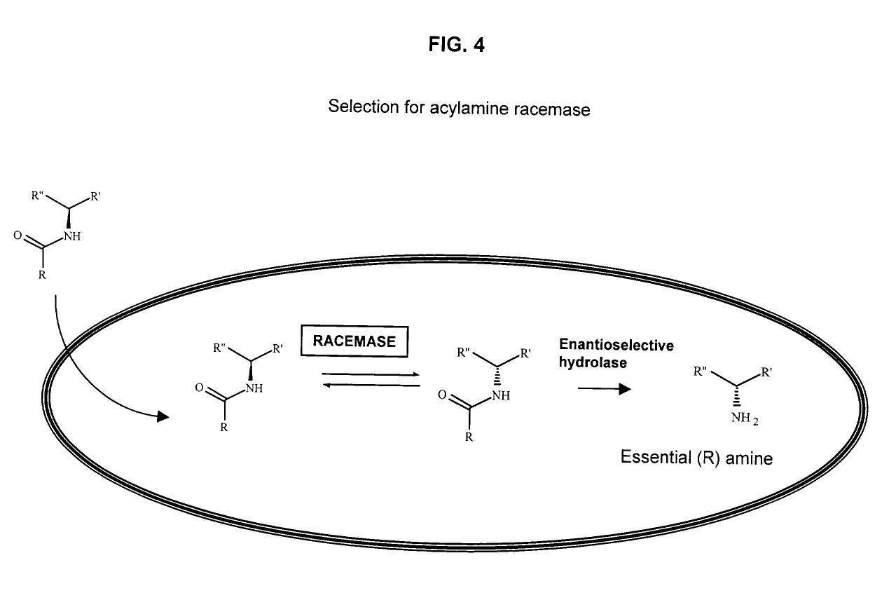

The validity of the microbial-based selection for acylamine racemases can be tested by addition to the growth medium of acyl derivatives of the S- or R-enantiomers of the essential amine for which the cell is auxotrophic. For example when the cell is deficient in an essential (S)-amine and the medium is supplemented with the acyl-(S)-amine the strain is able to hydrolyze the acyl-(S)-amine to release the required free amine and relieve the auxotrophy. When supplemented with the acyl-(R)-amine the strain requires a racemase activity to convert the acyl-(R)-amine to the acyl-(S)-amine to relieve the auxotrophy. This is illustrated in FIG. 3 for a deficiency in an essential (S)-amine and while in FIG. 4 the cell is deficient in a specific (R)-amine. Even if the strain can hydrolyze the opposite acyl isomer of the required amine it is unable to use the free amine to relieve the auxotrophy in the absence of an amine racemase.

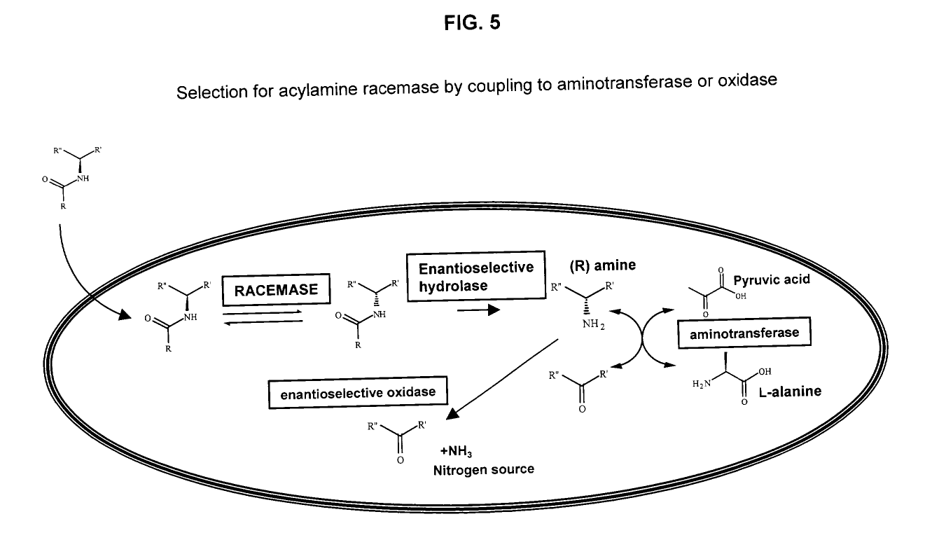

An additional aspect of the in vivo selection requires the introduction of an omega amino acid aminotransferase. Genes encoding such enzymes can be isolated from Bacilli and Pseudomonas by standard methods and expressed in E. coli. An omega amino acid aminotransferase can be used to enantioselectively transaminate one isomer of an amine into an essential amino acid for which the cell is auxotrophic thereby relieving the auxotrophy, thus identifying cells that have acylamine racemases. Similarly a gene encoding an enantioselective amine oxidase can be introduced to enable utilization of one enantiomer of a chiral amine as a sole nitrogen source for cell growth. This method to complement a deficiency in an amino acid using an omega amino acid aminotransferase or an amine oxidase specific for the (R)isomer of an amine is illustrated in FIG. 5.

The methods described in this invention can be used in bioprocesses to resolve racemic mixtures. N-acylamino acid racemases or acylamine racemases identified by the methods described above can be used in conjunction with enantioselective hydrolases to resolve racemic mixtures of derivatized amino acids or amines such that the reaction yield can approach the theoretical maximum of 100% by racemisation of unreacted substrate in situ. This is in contrast to processes which employ the enantioselective hydrolase activity alone which can only proceed to a theoretical maximum yield of 50% without other means of racemizing the unreacted substrate.

### EXAMPLES

### Example 1

### Construction of a Microbial Strain which Provides a Powerful Selective Method to Identify N-acylamino Acid Racemases

An amino acid auxotrophic mutant of Escherichia coli is obtained from a culture collection such as the Coli Genetic Stock Center, New Haven Conn. Such a mutant, for example E. coli CGSC 7421 or CGSC 7177 has an absolute requirement for supplemental amino acids when grown on a minimal salts medium such as M9 (Experiments in Molecular Genetics; Miller, Jeffrey H. (1972), 468 pp. Publisher:Cold Spring Harbor Lab., Cold Spring Harbor, N.Y.) with 0.2% glucose, 1 mM MgSO₄ 0.1 mM CaCl₂. In the case of CGSC 7421 the strain requires the growth medium to be supplemented with L-phenylalanine at 50 μg/ml. In the case of CGSC 7177 the strain requires the growth medium to be supplemented with L-methionine, L-arginine and L-tryptophan at 50 μg/ml. The strain is then additionally mutated in the dadA gene encoding D-amino acid oxidase. In the case of L-methionine auxotrophy, such a strain is also available (CGSC 6563). Alternatively, the dadA gene is deleted using methods generally available in the art (e.g. Datsenko and Wanner PNAS 2000, 97:6640-6645). This prevents the strain from relieving most auxotrophies by using D-amino acids as a source of L-amino acids. Using this method an internal deletion in the dadA gene of E. coli K12 can be prepared using the following oligonucleotide primers: 5′ AAC CAG TGC CGC GAA TGC CGG GCA AAT CTC CCC CGG ATA TGC TGC ACC GTC ATA TGA ATA TCC TCC TTA G-3′ and 5′-CCA CTA AAA CAG GGG TAC CGG TAG GCG CGT GGC GCG GAT AAC CGT CGG CGG TGT AGG CTG GAG CTG CTT CG-3′ with the pKD3 plasmid template described in the above reference. Plasmid pKD3 is available from the CGSC as part of the Gene Disruption Kit. The dadA deletion can be introduced to E. coli auxotrophs such as CGSC 7421 using methods standard in the art such as P1 transduction (Experiments in Molecular Genetics; Miller, Jeffrey H.. (1972), 468 pp. Publisher:Cold Spring Harbor Lab., Cold Spring Harbor, N.Y.). If necessary the mutant strain can be transformed with an expression vector which carries and expresses a gene encoding an aminoacylase specific for N-acyl-L-amino acids, such as the ama gene of B. stearothermophilus, (Applied and Environmental Microbiology 1993, 59:3878-3888). The resulting transformant is then more capable of hydrolyzing supplemental acylated L-amino acids such as N-acetyl-L-amino acids to generate the free amino acid. This enables acylated L-amino acids to be the source of the deficient L-amino acid necessary to relieve the auxotrophy and enable growth of the organism on the minimal medium. The medium can be further modified to select further racemase variants with industrially desirable properties, for example by elimination or substitution of the cobalt in the medium to identify racemase variants with reduced or eliminated requirement for cobalt.

Such a strain can then be grown on a minimal medium which establishes a selective pressure for an N-acylamino acid racemase activity. When the medium is supplemented with an N-acyl-D-amino acid instead of a required L-amino acid, such as N-acetyl-D-phenylalanine in the case of a CAG112158/dadA mutant (ET2) or N-acetyl-D-methionine in the case of CGSC6563 then the organism cannot use this substrate as a source of the deficient amino acid. As a result a selective pressure is established for an enzymatic racemase activity capable of racemising the N acyl-D-amino acid to a mixture of the N acyl-D-amino acid and the corresponding N-acyl-L-amino acid. The latter can then be hydrolyzed by cellular hydrolases to provide the amino acid necessary to relieve the auxotrophy and enable growth of the organism on the minimal medium. In this way the amino acid auxotrophic/dadA mutant can be used to identify additional genes introduced to the strain which encode and express enzymes with N-acylamino acid racemase activity.

This was demonstrated by the introduction of a gene encoding a known N-acylamino acid racemase activity such as TS-1-60 (Appl. Microbiol. Biotechnol. 42:884-889). The gene encoding this racemase can be isolated from the chromosomal DNA of strain IFO 15079 (IFO Osaka, Japan)by PCR using standard conditions and the following oligonucleotide primers: 5′-GAC AGG ACG AAT TCG AAA CTC AGC GGT GTG GAA CTG CGC CGG G-3′ and 5′-GAC GCA TGC CTA CGA ACC GAT CCA CAC CTT TGC CGT GGT C-3′. The fragment generated was cleaved by EcoRI and SphI and ligated to the large fragment of similarly cleaved expression vector pTTQ18 (Dr. M. Stark, Dundee University) to generate plasmid pSR7-1. Many other expression vectors such as those available from Invitrogen (Carlsbad, Calif.) would also be equivalent. Expression of the gene on pTTQ18 can then be induced by addition of 1 mM IPTG to the growth medium. Cells of CGSC6563 transformed by pSR7-1 can be grown on a racemase selective agar medium prepared as follows: To 270 ml of autoclaved 1.5% water agar cooled to 50° C. add: 300 μl of MgSO₄ (1 M), 30 μl of CaCl₂ (1M), 3 ml of 20% dextrose, 30 ml of M9 salts, 3 ml of L-Arg (5 mg/ml), 3 ml of L-Trp (5 mg/ml), 3 ml of N-Acetyl D-Methionine (5 mg/ml), 24 mg of CoCl₂ dissolved into water (final concentration in medium of 0.3 mM), 600 μl of Ampicilin (100 mg/ml), 73 mg of IPTG dissolved into water. Colonies of CGSC6563/pRS7-1 can be grown on this medium following incubation at 37° C. for 96 hrs. A control strain comprising CGSC6563/pTTQ18 (i.e., with no cloned gene encoding racemase activity) shows no growth upon this medium.

### Example 2

### Identification of a Novel Racemase Capable of Racemising N-acylamino Acids from Microbial Genomic Libraries

A strain such as that described in Example 1 can be transformed with an expression plasmid or phagemid library containing and expressing random fragments of chromosomal DNA derived from microbial sources such as microbial culture collections or soil organisms. Such organisms might include but are not limited to Actinomycetes, Pseudomonads, Bacillus, Rhodococci, Escherichia, Salmonella, Klebsiella. The presence of genes from such libraries encoding N-acylamino acid racemases would be detected by growth of individual colonies transformed with members of the plasmid library which carry and express such genes when plated on minimal medium lacking the necessary amino acid supplement to relieve the auxotrophy of the strain and when supplemented with the appropriate N-acylated amino acid enantiomer.

### Example 3

### Identification of a Novel Racemase Capable of Racemising N-acylamino Acids from a Member of the Enolase Superfamily of Enzymes

A strain such as that described in Example 1 can be transformed with an expression plasmid containing and expressing a gene or genes encoding members of the enolase superfamily of enzymes (Babbitt, P. C. et al 1996 Biochemistry 35:16489-16501). Such enzymes possess the common ability to catalyze the abstraction of the α-proton of a carboxylic acid to form an enolic intermediate. The presence of genes encoding and expressing members of the enolase superfamily of enzymes which possess N-acylamino acid racemase activity would be detected by growth of individual colonies transformed with a plasmid which carries and expresses such genes when plated on minimal medium lacking the necessary amino acid supplement to relieve the auxotrophy of the strain and when supplemented with the appropriate N-acylated amino acid enantiomer.

### Example 4

### Identification of a Novel Racemase Capable of Racemising N-acylamino Acids by Directed Evolution from a Member of the Enolase Superfamily of Enzymes

A strain such as that described in Example 1 can be transformed with an expression plasmid containing and expressing a gene or genes encoding members of the enolase superfamily of enzymes. Such enzymes possess the common ability to catalyze the abstraction of the α-proton of a carboxylic acid to form an enolic intermediate. The gene or genes encoding enzymes of the enolase superfamily of enzymes can first be subjected to random mutagenesis using methods well known in the art such as chemical (e.g. NTG), biological (mutator strain, error prone PCR) or other (e.g. UV irradiation) methods. The presence of genes or gene variants which encode and express members of the enolase superfamily of enzymes which possess N-acylamino acid racemase activity would be detected by growth of individual colonies transformed with a plasmid which carries and expresses such genes when plated on minimal medium lacking the necessary amino acid supplement to relieve the auxotrophy of the strain and when supplemented with the appropriate N-acylated amino acid enantiomer.

### Example 5

### Construction of a Microbial Strain which Provides a Powerful Selective Method to Identify Acylamine Racemases

A strain of Escherichia coli such as E.coli K12 W3110 or specific mutants thereof is obtained from a culture collection such as the Coli Genetic Stock Center. The strain is grown on a minimal salts medium without a nitrogen source. Such a medium could be M9 minimal salts which lacks the ammonia component. The media is supplemented with 0.2% glucose, 1 mM MgSO₄ and 0.1 mM CaCl₂. The strain is transformed with an expression plasmid which expresses an enzyme capable of eliminating ammonia or transferring an amino group selectively from one enantiomer of a chiral amine. The liberated ammonia can be used as a source of nitrogen by the organism, enabling growth on a selective medium with no alternate source of nitrogen. Such an enzyme could be an amine oxidase or an omega amino acid aminotransferase which are known to act in an enantioselective way to eliminate or transfer nitrogen in the form of ammonia or an amino group from amines. Such a strain can be co-transformed with a gene encoding an enantioselective hydrolase capable of liberating the amine substrate of the oxidase or aminotransferase from a derivatized precursor. The resulting transformant can be used to select for an enzymatic amine racemase activity in the following way. The strain is provided with a nitrogen free growth medium, supplemented with the opposite enantiomer of for example an acylated amine substrate of an acylase or amidase. This provides a selection for a racemase activity which converts the supplemental acylated amine to a mixture of both enantiomers. The resulting racemate can then serve as a substrate for the acylase or amidase and the nitrogen can be made accessible to the cell by the transaminase or oxidase. This results in selective cell growth of a strain which possesses acylamine racemase activity.

### Example 6

### Identification of a Novel Racemase Capable of Racemising Acylamines from Microbial Genomic Libraries

A strain such as that described in Example 5 can be transformed with an expression plasmid or phagemid library containing and expressing random fragments of chromosomal DNA derived from microbial sources such as microbial culture collections or soil organisms. Such organisms might include but are not limited to Actinomycetes, Pseudomonads, Bacillus, Rhodococci, Escherichia, Salmonella, Klebsiella. The presence of genes from such libraries encoding acylamine racemases would be detected by growth of individual colonies transformed with members of the plasmid library which carry and express such genes when plated on minimal medium lacking an alternate nitrogen source and when supplemented with the appropriate enantiomer of an acylated amine.

### Example 7

### Identification of a Novel Racemase Capable of Racemising Acylamines from a Member of the Enolase Superfamily of Enzymes

A strain such as that described in Example 1 can be transformed with an expression plasmid containing and expressing a gene or genes encoding members of the enolase superfamily of enzymes. Such enzymes possess the common ability to catalyze the abstraction of the α-proton of a carboxylic acid to form an enolic intermediate. The presence of genes encoding and expressing members of the enolase superfamily of enzymes which possess N-acylamino acid racemase activity would be detected by growth of individual colonies transformed with a plasmid which carries and expresses such genes when plated on minimal medium lacking an alternate nitrogen source and when supplemented with the appropriate enantiomer of an acylated amine.

### Example 8

### Identification of a Novel Racemase Capable of Racemising Acylamines by Directed Evolution from a Member of the Enolase Superfamily of Enzymes

A strain such as that described in Example 1 can be transformed with an expression plasmid containing and expressing a gene or genes encoding members of the enolase superfamily of enzymes. Such enzymes possess the common ability to catalyze the abstraction of the α-proton of a carboxylic acid to form an enolic intermediate. The gene or genes encoding enzymes of the enolase superfamily of enzymes can first be subjected to random mutagenesis using chemical (e.g. NTG), biological (mutator strain, error prone PCR) or other (e.g. UV irradiation) methods. The presence of genes or gene variants which encode and express members of the enolase superfamily of enzymes which possess N-acylamino acid racemase activity would be detected by growth of individual colonies transformed with a plasmid which carries and expresses such genes when plated on minimal medium lacking an alternate nitrogen source and when supplemented with the appropriate enantiomer of an acylated amine.

1. A method of identifying an enzyme from the enolase superfamily which has acquired racemase activity comprising:
a) obtaining a micro-organism which requires the enzyme with the acquired racemase activity to grow in a selective medium,
b) transforming the micro-organism with a gene that encodes the enzyme,
c) growing the transformed micro-organism in the selective medium supplemented with a substrate of the racemase,
d) selecting one or more micro-organism(s) that are able to grow in the supplemented selective medium, thereby identifying the enzyme with the acquired racemase activity.

2. The method according to claim 1, wherein the enzyme acquires a racemase activity due to one or more genetic alterations.

3. The method according to claim 2, wherein the genetic alterations results in the enzyme acquiring a racemase activity similar to another member of the enolase superfamily.

4. The method according to claim 1 wherein the selective medium does not contain cobalt.

5. The method according to claim 1 wherein the selective medium contains a metal that is able to substitute for cobalt.

6. The method according to claim 1 wherein the racemase activity is N-acyl-amino acid racemase or acylamine racemase.

7. The method according to claim 1 wherein the enzyme acquires N-acylamino acid racemase activity when the substrate is an N-acylamino acid.

8. The method according to claim 1 wherein the enzyme acquires acylamine racemase activity when the substrate is an acylamine.

9. The method according to claim 2 wherein the genetic alterations are obtained by site-directed mutagenesis based on identification of conservation of amino acid sequence, structure, and enzymatic mechanism.

10. The micro-organism according to claim 1 which possesses one or more mutations which create one or more amino acid auxotrophies and a deficiency in D-amino acid oxidase activity.

11. A method to enantioselectively produce an L-amino acid or a D-amino acid comprising combining a hydrolytic enzyme with the racemase identified according to claim 1.

12. The method according to claim 11 wherein the hydrolytic enzyme is selected from the group consisting of amidase or acylase.

13. A bacterial strain transformed with a vector carrying a gene encoding an enzyme having N-acylamino acid racemase activity identified according to the method of claim 1.

14. The bacterial strain according to claim 13 that is able to express the enzyme.

15. A gene encoding an enzyme with N-acylamino acid racemase activity identified by the method of claim 1.

16. The gene according to claim 15 wherein the gene is derived from a gene library prepared from the chromosome of a micro-organism.

17. The gene library according to claim 16 wherein the library has been subjected to a method of random genetic mutation.

18. The method of random genetic mutation according to claim 17 comprising passage through a mutator strain of E. coli.

19. The method of random genetic mutation according to claim 17 comprising error prone PCR.

20. The mutator strain of E. coli according to claim 18 that is XL1-Red.

21. The micro-organism according to claim 16 selected from the group consisting of Bacillus, Actinomyces, Rhodococcus, Escherichia, Salmonella, Klebsiella, and Pseudomonas.

22. The gene according to claim 15 wherein the gene is derived from one or more genes encoding one or more members of the enolase enzyme superfamily.

23. An enzyme which possesses N-acylamino acid racemase activity identified according to the method of claim 1.

24. A vector which carries a gene encoding an enzyme having N-acylamino acid racemase activity identified by the method of claim 1.

25. A recombinant bacterial strain comprising the following characteristics:
a) auxotrophic for a specific enantiomer of one or more amino compounds,
b) able to preferentially hydrolyze the acyl derivatives of the amino compounds for which the bacterial strain is auxotrophic,
c) able to express an enzyme comprising racemase activity from a gene fragment carried on a vector to racemize the acyl derivatives of the amino compounds,
d) able to couple the racemase activity of the expressed enzyme with hydrolytic enzymes to produce a single enantiomer of the amino compounds from an acyl racemic mixture of the amino compounds, and
e) able to grow in selective medium that lacks the specific enantiomer of the amino compounds for which it is auxotrophic.

26. An enzymatic process for enantioselectively producing an L-amino acid or a D-amino acid comprising combining a hydrolytic enzyme with a racemase identified according to the method of claim 1.

27. The hydrolytic enzyme of claim 26 selected from the group consisting of amidase and acylase.

28. The enzymatic process according to claim 26 wherein one or more enzymes are immobilized.

29. A screening method to identify novel racemase activity derived by mutation of one or more genes encoding a member or members of the enolase superfamily of enzymes comprising:
a) mutation of the gene,
b) transformation of a bacterial strain with a plasmid bearing the mutated gene, wherein the bacterial strain requires the product of novel racemase activity for growth on selective media, and
c) selecting the bacterial strains that are capable of growing in a selective medium that lacks the product of the racemase.

30. The screening method of claim 29 wherein the mutated gene encodes an enzyme with N-acylamino acid racemase activity.

31. The screening method of claim 29 wherein the mutated gene encodes an enzyme with acylamine racemase activity.
